(19) United States
(12) Patent Application Publication
(10) Pub. No.: US 2007/0148710 A1
(43) Pub. Date: Jun. 28, 2007

(54) Cpt resistant cell line

(76) Inventor: Donald J. Buchsbaum, Alabaster, AL (US)

Correspondence Address: FISH & RICHARDSON P.C., P.O BOX 1022, Minneapolis, MN, 55440-1022, US

(21) Appl. No.: 10/571,453

(22) PCT Filed: Sep. 10, 2004
(86) PCT No.: PCT/US04/29696

§ 371 Date: Aug. 21, 2006

Related U.S. Application Data
(60) Provisional application No. 60/502,743, filed on Sep. 12, 2003.

(51) Int. Cl. G01N 33/574; C12N 5/08

(52) U.S. Cl. 435/7.23; 435/325; 435/366

(57) ABSTRACT

Disclosed are compositions relating to cells and cell lines that are resistant to a given chemotherapeutics as well as method for making and using the same.

### I. BACKGROUND OF THE INVENTION

1. Chemoresistance is a problem in treating subjects with cancer. Sustained exposure to chemotherapeutics can result in a resistance to the positive effects of the chemotherapeutic agents. Such resistance results in proliferation of the cancer cells and often necessitates changes in treatment. Needed in the art is a means of reversing chemoresistance.

II. SUMMARY OF THE INVENTION

2. In accordance with the purposes of this invention, as embodied and broadly described herein, this invention, in one aspect, relates to an isolated cell and a stable cell line that possess resistance to CPT or a derivative or a metabolite thereof. In another aspect, this invention relates to an isolated cell and a stable cell line that possess resistance to CPT or a derivative or a metabolite thereof and resistance to at least one additional chemotherapeutic agent. Also provided herein are methods of making and using such cells and cell lines and methods of reducing or reversing chemoresistance.

3. Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### III. BRIEF DESCRIPTION OF THE DRAWINGS

4. The accompanying drawings, which are incorporated in and constitute a part of this specification, illustrate several embodiments of the invention and together with the description, serve to explain the principles of the invention.

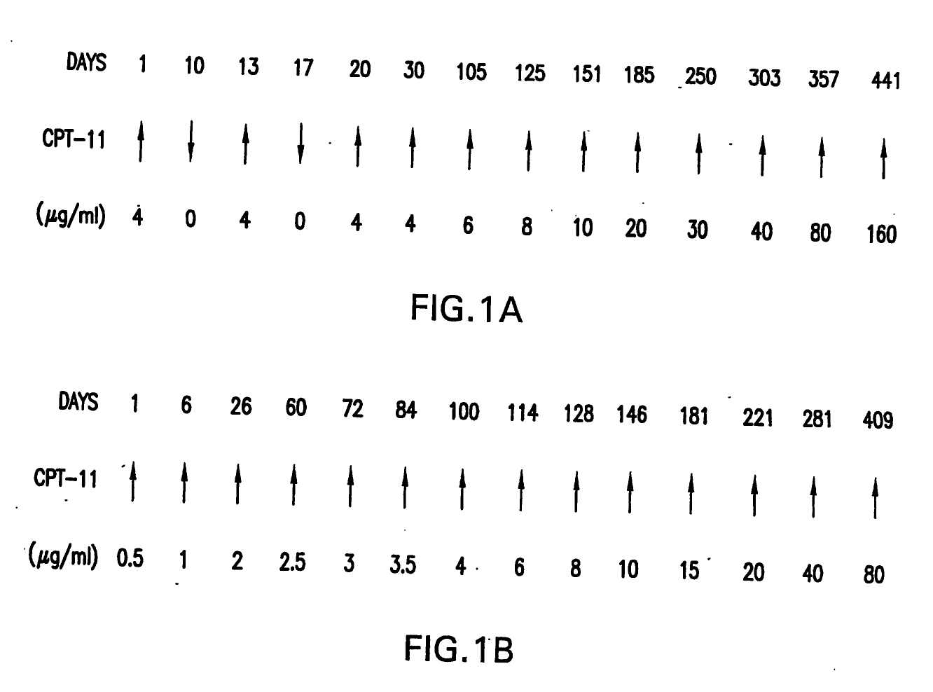

5. FIG. 1 shows the dose schema for promoting of CPT-11 (Irinotecan) resistance. FIG. 1A shows the generation of SW948CPTH cell line. FIG. 1B shows the generation of SW948CPTL cell line. Up arrows indicate addition of CPT-11 to the culture medium, and down arrows indicate removal of CPT-11 from the culture medium. Days indicate the time at which CPT-11 concentrations were changed.

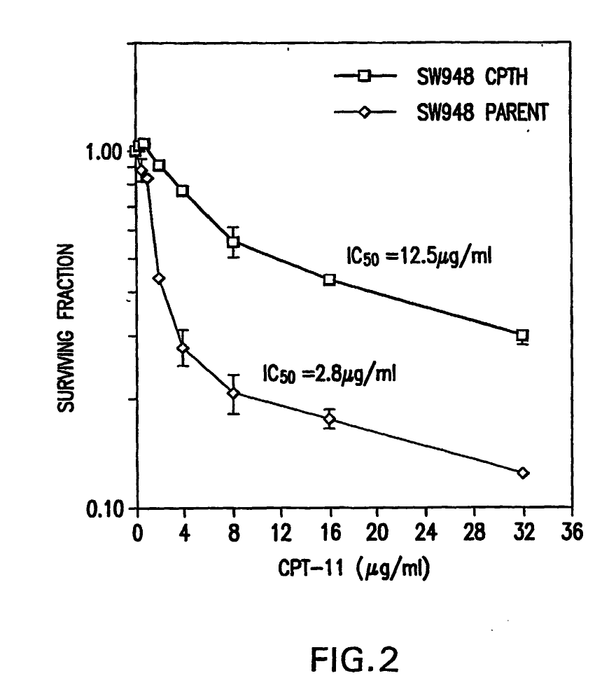

6. FIG. 2 shows the CPT-11 inhibitory concentration for 50% colony formation (IC₅₀) of SW948CPTH cells and the parental SW948 cells in a clonogenic cell survival assay.

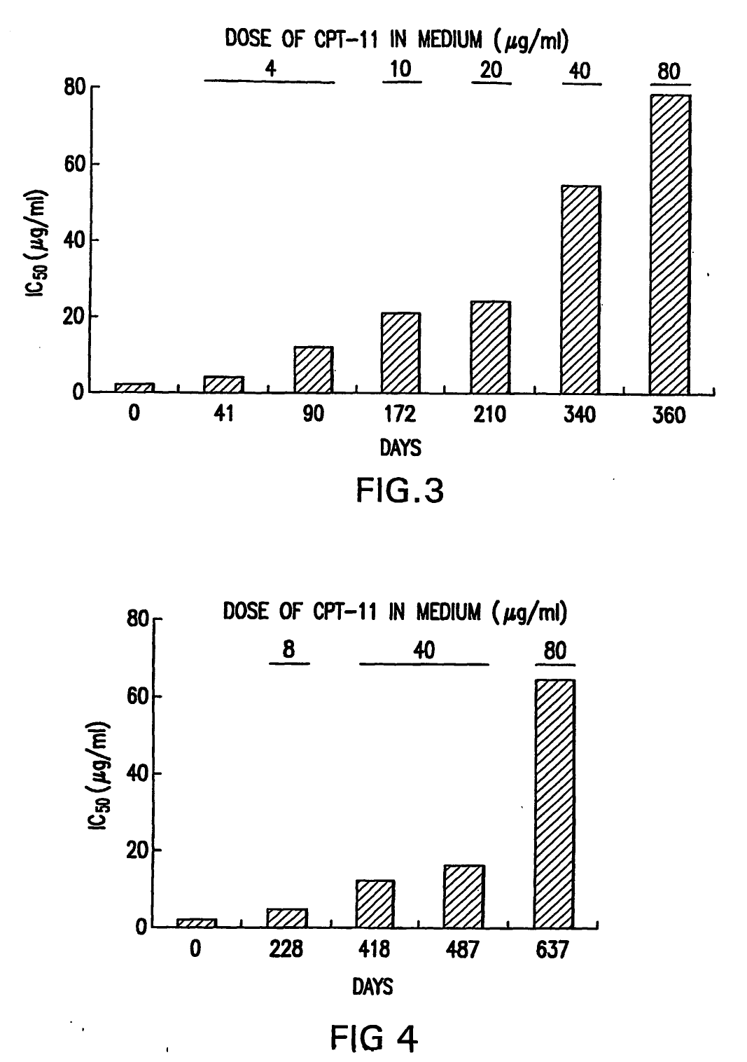

7. FIG. 3 shows the CPT-11 IC₅₀ values of SW948CPTH cells during the course of exposure to CPT-11. IC₅₀ was determined using a clonogenic assay.

8. FIG. 4 shows the CPT-11 IC₅₀ values of SW948CPTL cells during the course of exposure to CPT-11. IC₅₀ was determined using a clonogenic assay.

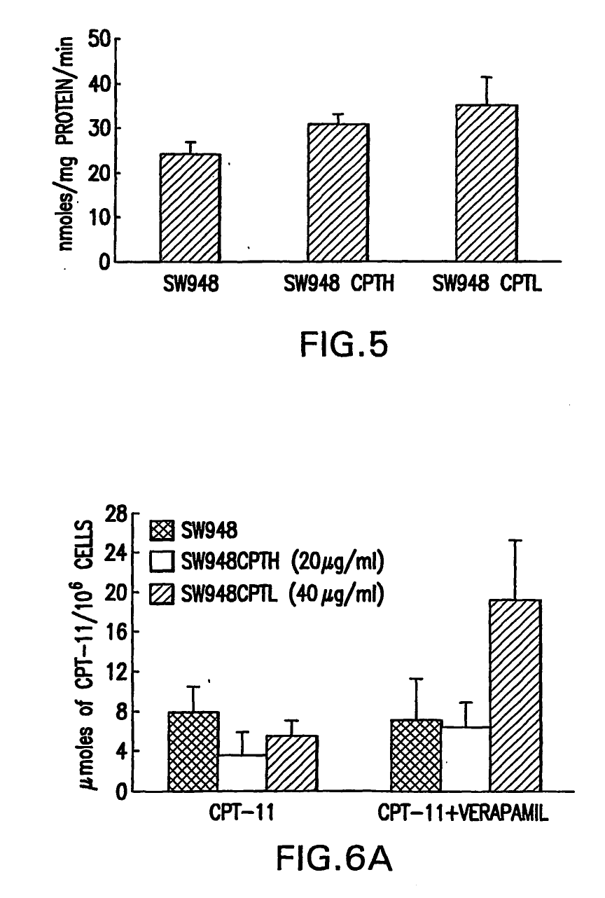

9. FIG. 5 shows the carboxyesterase activity measurements in SW948, SW948CPTH, and CS948CPTL cell lines. SW948CPTH cells were treated with CPT-11 for 156 days at 20 μg/ml and cultured without CPT-11 for 37 days. SW948CPTL cells were treated with CPT-11 for 44 days and cultured without CPT-11 for 49 days. Results are expressed as mean±SD.

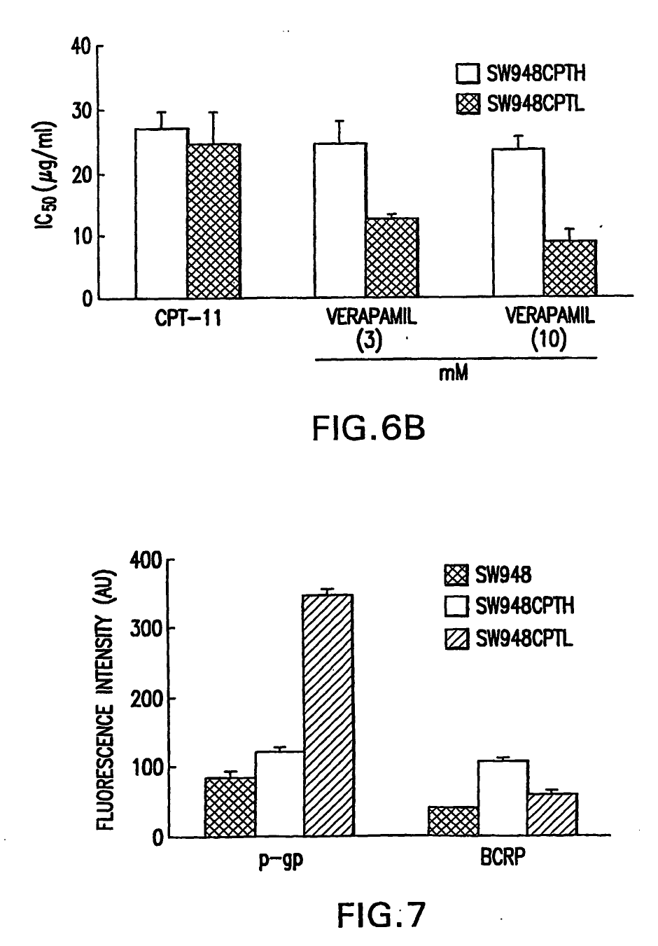

10. FIG. 6A shows the intracellular accumulation of CPT-11 and the effects of verapamil: SW948, SW948CPTH, and SW948CPTL cells were incubated with CPT-11, 160 μM, for 2 hours at 37° C. in the presence or absence of 10 μM verapamil. Cells were washed, trypsinized, and counted. Cell pellets were lysed with water and sonicated. The amounts of CPT-11 in the supernatant harvested from the samples were determined with HPLC analysis. The values of CPT-11 were calculated as μmoles/10⁶ cells. Results were generated from three independent experiments. In two experiments, SW948CPTH cells were treated with CPT-11, 40 μg/ml for 36 days and cultured without CPT-11 for 32 and 39 days, respectively. In another experiment using SW948CPTH, cells were treated with CPT-11, 20 μg/ml for 58 days and cultured without CPT-11 for 12 days. SW948CPTL cells were treated with CPT-11, 40 μg/ml for 46 days and cultured without CPT-11 for 13, 30 and 45 days respectively. Data are expressed as mean±SD. FIG. 6B shows the effect of verapamil on the CPT-11 IC₅₀ value of SW948CPTH and SW948CPTL cells. Cells were incubated with CPT-11 for 24 hours at 37° C. in the presence or absence of verapamil (3 or 10 μM), and the CPT-11 IC₅₀ value determined by clonogenic assay. Data are the mean±SD of two experiments done in triplicate.

11. FIG. 7 shows the level of p-glycoprotein (p-gp) and breast cancer resistant protein (BCRP) in SW948, SW948CPTH, and SW948CPTL cells. SW948CPTH cells were cultured in medium containing CPT-11, 20 μg/ml for 146 days and subsequently cultured without CPT-11 for 21 days. SW948CPTL cells were cultured in medium containing CPT-11, 40 μg/ml for 45 days and subsequently cultured without CPT-11 for 7 days. Cells were washed with phosphate buffered saline (PBS), scraped, and aliquoted into 12×75 mm tubes. Cells were incubated with mouse antibodies against p-glycoprotein (p-gp) or breast cancer resistant protein (BCRP) for 30 min at 40° C., washed with PBS/1% BSA followed by FITC-conjugated goat anti-mouse antibody. Fluorescence intensity of the cells was monitored and analyzed by flow cytometry after further washing. Data are shown as mean fluorescence intensity±SD from triplicates.

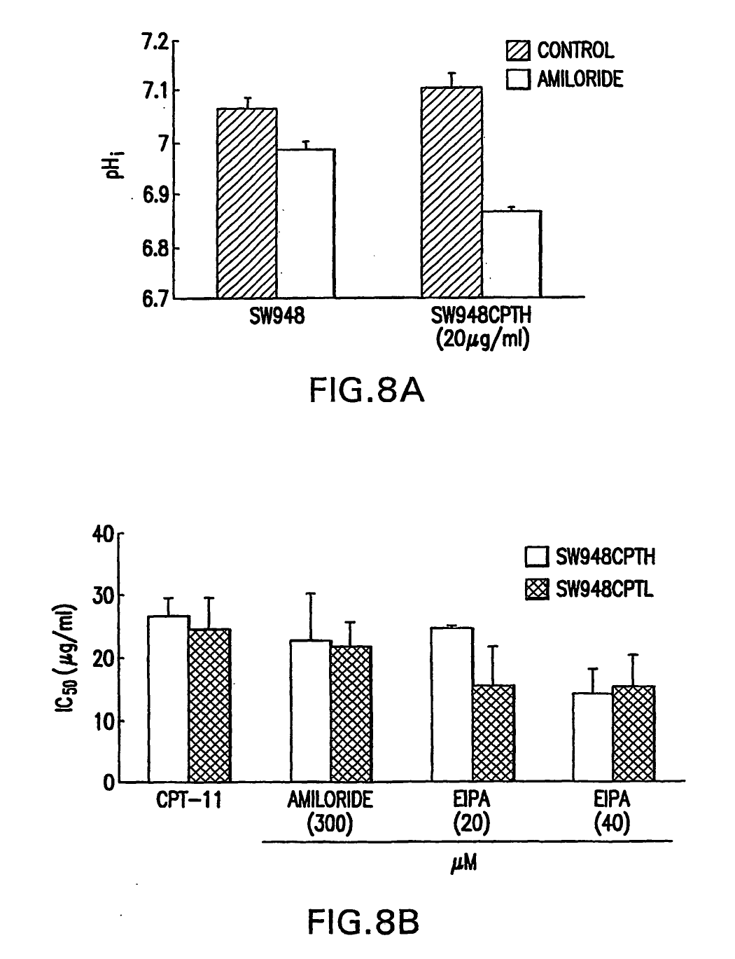

12. FIG. 8A shows the intracellular pH (pHi) of SW948 and SW948CPTH cells in the presence or absence of amiloride. SW948CPTH cells were treated with CPT-11 at 20 μg/ml for 160 days and cultured without CPT-11 for 84 days. Cells were plated on glass coverslips and cultured overnight. Intracellular pH was measured with a fluorescence spectrophotometer after cells were loaded with the pH-sensitive fluorescence dye, BCECF. The data represent the mean±SD of 60 individual cells. FIG. 8B shows the change in CPT-11 IC₅₀ values after exposure of SW948CPTH and SW948CPTL cells to CPT-11 and amiloride or its derivative 5-(N-ethyl-N-isopropyl)amiloride (EIPA). SW948CPTH cells were treated with CPT-11 at 20 μg/ml for 156 days and cultured without CPT-11 for 31 or 58 days respectively. SW948CPTL cells were treated with CPT-11 at 40 μg/ml for 44 days and cultured without CPT-11 for 54 days. The IC₅₀ values were determined using a clonogenic assay in which amiloride (300 μM) or its derivative, EIPA (3 or 10 μM) was added to the culture medium followed immediately by CPT-11 (0-64 μg/ml). After 24 h the drugs were removed, cells washed with PBS and drug-free culture medium added to the cell cultures. Data represent the mean±SD from three assays done in triplicate with SW948CPTH cells and two assays done in triplicate for SW948CPTL cells.

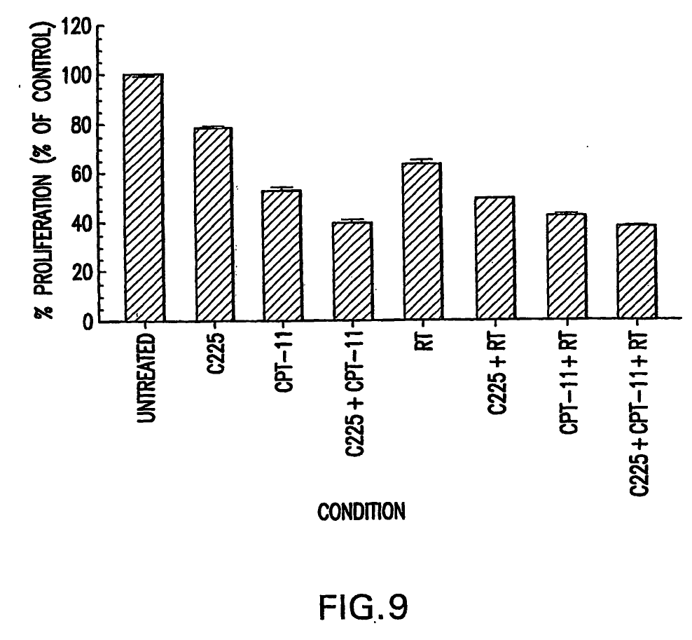

13. FIG. 9 shows in vitro inhibition of SW948 colon carcinoma cell growth. SW948 cells were treated with Erbitux® [5 μg/ml] on day 0, CPT-11 [3 μg/ml] on day 1 for 24 hours, followed by 2 Gy ⁶⁰Co irradiation (RT) on day 2, or the combination of each agent, then cells counted on day 4. Data points are the average±SEM of three independent experiments, each done in quadruplicate cultures (n=12), then normalized to the untreated control (100%).

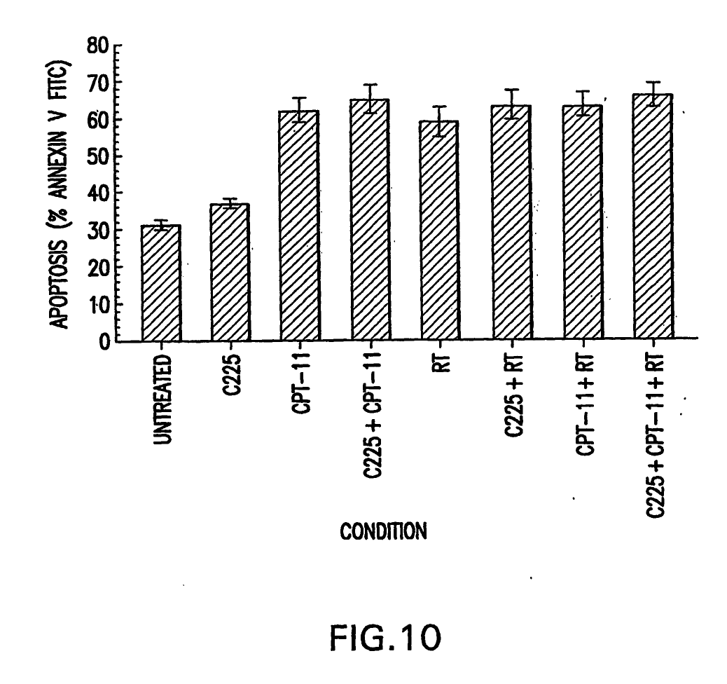

14. FIG. 10 shows the analysis of SW948 colon carcinoma cells for the induction of early apoptosis. Floating and adherent cells were collected 4 days post-treatment. The treatments are described in the legend for FIG. 8. The cells were stained with annexin V-FITC and propidium iodide, then analyzed by FACS using CellQuest software (Beckton-Dickenson, San Diego, Calif.). Data points are the average±SEM of a representative experiment done in triplicate.

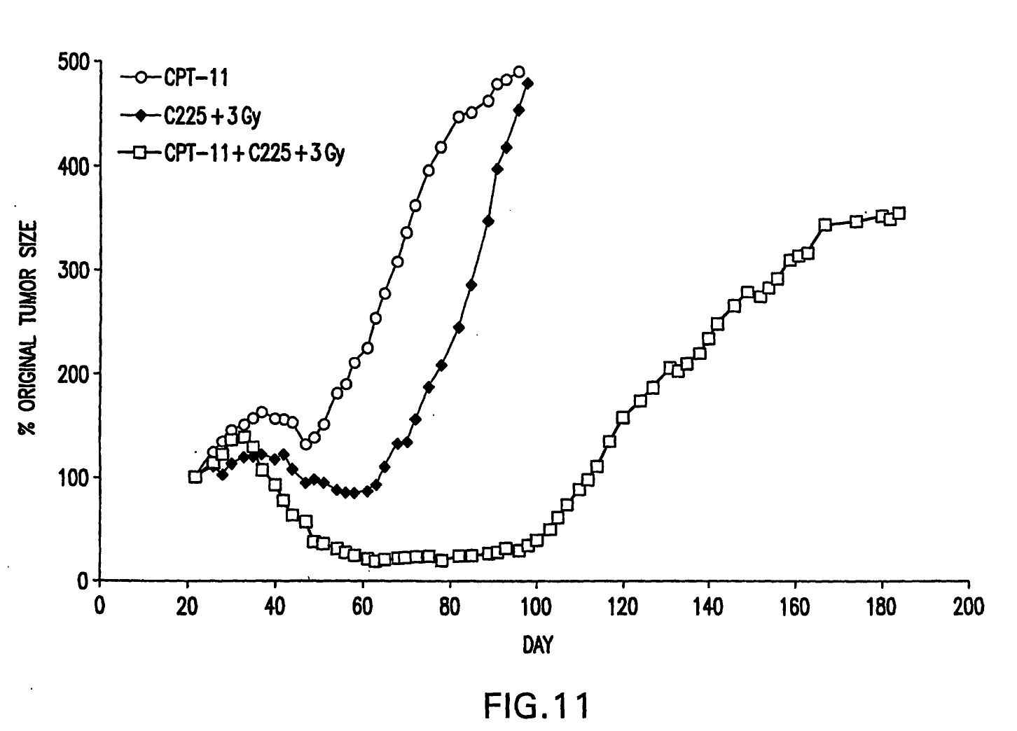

15. FIG. 11 shows the effect of Erbitux®, CPT-11, and radiation alone or in combination on the growth of SW948 human colon cancer xenografts. Erbitux® (1 mg) was administered i.p. every 3 days for 5 weeks beginning on day 22 after tumor cell injection. CPT-11 (40 mg/kg) was administered i.v. on days 23, 29, 35, 41, 47, and 55 after tumor cell injection. Tumors received 3 Gy ⁶⁰Co radiation 1 hour after each injection of CPT-11. Mean change in tumor size relative to size on day 22 (n=7 mice/group) is shown. At the time of Erbitux® administration (day 22), the mean±SD size of the tumors were 71.8±39.8 mm².

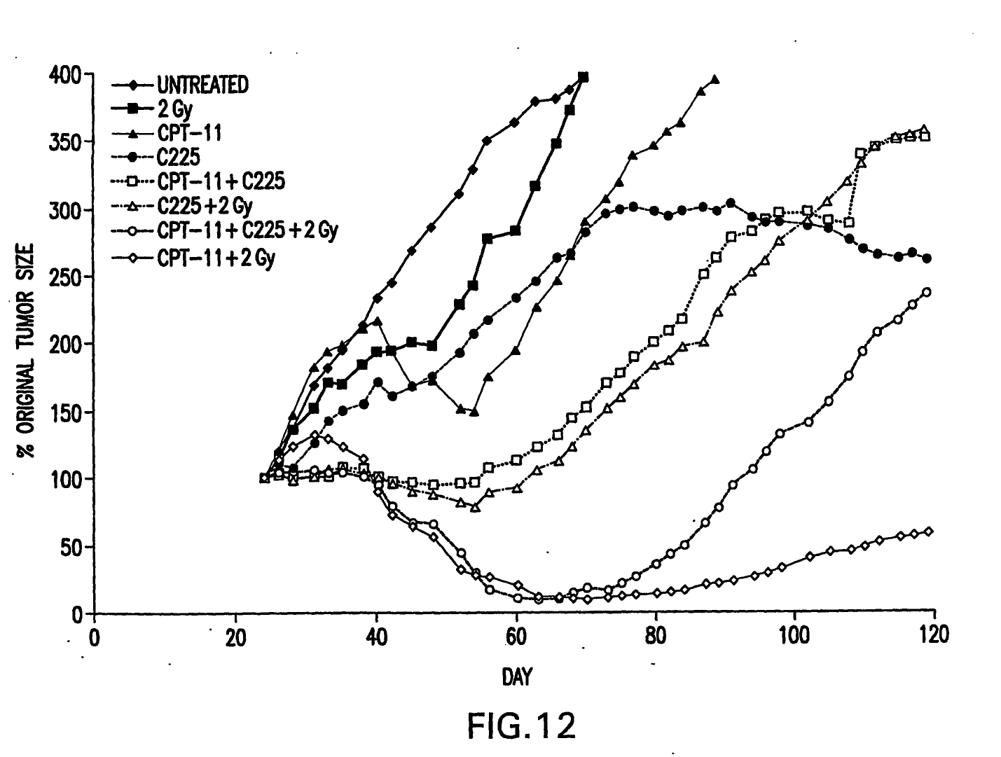

16. FIG. 12 shows the effect of Erbitux®, CPT-11, and radiation alone or in combination on the growth of SW948 human colon cancer xenografts. Erbitux® (1 mg) was administered i.p. 2 times a week for 3 weeks (days 24, 27, 31, 34, 38, and 41) beginning on day 24 after tumor cell injection. CPT-11 (25 mg/kg) was administered i.v. on days 25, 28, 32, 35, 39, and 42 after tumor cell injection. Tumors received 2 Gy ⁶⁰Co radiation 1 hour after each injection of CPT-11. Mean change in tumor size relative to size on day 24 (n=7 mice/group). At the time of Erbitux® administration (day 24), the mean±SD size of the tumors were 58.0±29.3 mm².

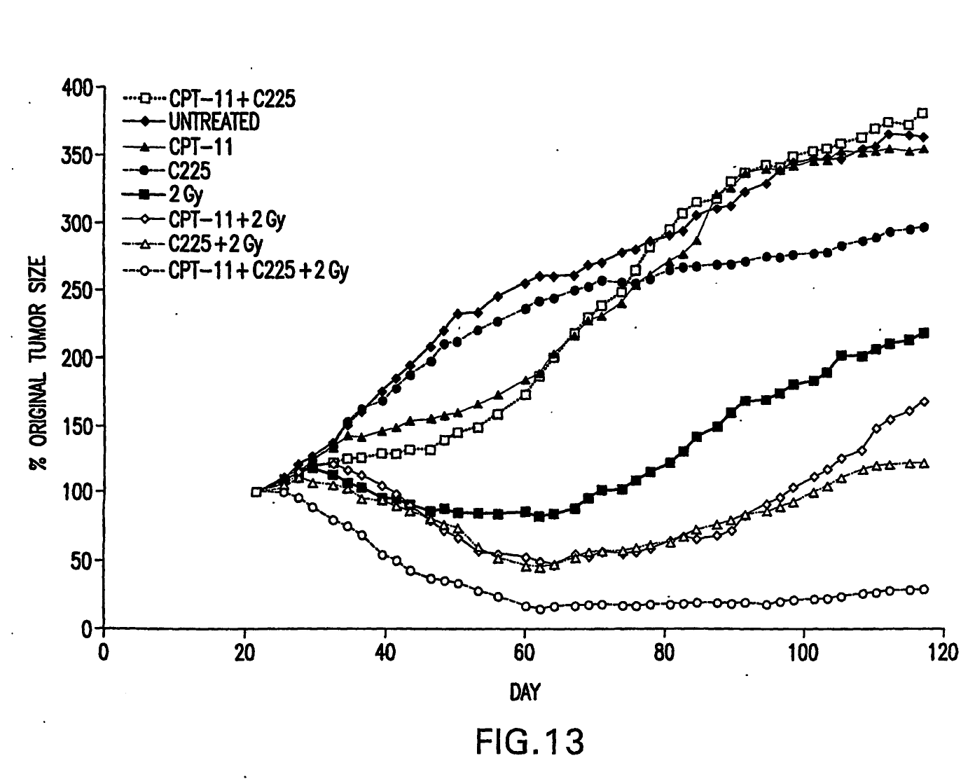

17. FIG. 13 shows the effect of Erbitux®, CPT-11, and radiation alone or in combination on the growth of SW948CPTH human colon cancer xenografts. The SW948CPTH cells were grown in the presence of 20 μg/ml CPT-11, then the drug removed for 7 days prior to implantation into the mice. Erbitux® (1 mg) was administered i.p. 2 times a week for 3 weeks (days 22, 26, 29, 33, 36, and 40) beginning on day 22 after tumor cell injection. CPT-11 (25 mg/kg) was administered i.v. on days 23, 27, 30, 34, 37, and 41 after tumor cell injection. Tumors received 2 Gy ⁶⁰Co radiation 1 hour after each injection of CPT-11. Mean change in tumor size relative to size on day 22 (n=7 mice/group). At the time of Erbitux® administration (day 22), the mean±SD size of the tumors were 62.6±26.6 mm².

### IV. DETAILED DESCRIPTION

18. The present invention may be understood more readily by reference to the following detailed description of preferred embodiments of the invention and the Examples included therein and to the Figures and their previous and following description.

19. Before the present compounds, compositions, articles, devices, and/or methods are disclosed and described, it is to be understood that this invention is not limited to specific synthetic methods, specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

A. Definitions

20. As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

21. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

22. In this specification and in the claims which follow, reference will be made to a number of terms which shall be defined to have the following meanings:

23. “Optional” or “optionally” means that the subsequently described event or circumstance may or may not occur, and that the description includes instances where said event or circumstance occurs and instances where it does not.

B. Compositions and Methods

1. Compositions

24. Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed, that while specific reference of each various individual and collective combination and permutation of these compounds may not be explicitly disclosed, each is specifically contemplated and described herein. For example, if a particular CPT-11 Irinotecan) resistant cell or cell line is disclosed and discussed and a number of modifications that can be made to a number of molecules including a CPT-11 resistant cell or cell line are discussed, specifically contemplated is each and every combination and permutation in a particular CPT-11 resistant cell or cell line and the modifications that are possible, unless specifically indicated to the contrary. Thus, if a class of molecules A, B, and C is disclosed as well as a class of molecules D, E, and F, and if an example of a combination molecule (e.g., A-D) is disclosed, then even if each combination is not individually recited, each possible combination is individually and collectively contemplated. Therefore, for example, combinations A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there is a variety of additional steps that can be performed, it is understood that each of these additional steps can be performed with any specific embodiment or combination of embodiments of the disclosed methods.

25. The invention described herein relates in one aspect to a stable cell or cell line that is resistant to a chemotherapeutic. For example, specifically disclosed is a cancer cell line resistant to the chemotherapeutic CPT or a derivative or a metabolite thereof. Thus, one aspect of the present invention is a stable cell line resistant to CPT or a derivative or a metabolite thereof Also disclosed are cells from a cell line resistant to CPT or a derivative or a metabolite thereof. Examples of derivatives of CPT include, but are not limited to, CPT-11 and 10-OH-CPT. An example of a metabolite of CPT is SN38. Also provided herein is a cancer cell line resistant to CPT or a derivative or a metabolite thereof and at least one additional chemotherapeutic agent. Thus, also disclosed are cells from a cell line resistant to CPT or a derivative or a metabolite thereof and at least one additional chemotherapeutic agent. For example, specifically disclosed is a cancer cell line resistant to the chemotherapeutic CPT-11. Thus, the present invention provides a stable CPT-11 resistant cell line. Also disclosed are cells from a CPT-11 resistant cell line. The cancer cell line resistant to CPT-11 and at least one additional chemotherapeutic agent optionally shows cross resistance to at least one chemotherapeutic agent selected from the group consisting of actinomycin D, camptothecin, capecitabine, carboplatin, cisplatin, colchicine, daunorubicin, docetaxel, doxorubicin, epirubicin, etoposide, 5-fluorouracil, gemcitabine, melphalan, methotrexate, mitomycin C, mitoxantrone, paclitaxel, topotecan, vinblastine, and vincristine. As used herein, a “CPT resistant cell” and a “CPT resistant cell line” can include a cell or cell line that is resistant to CPT or a derivative or a metabolite thereof and at least one additional chemotherapeutic agent. It is understood that the cancer cell line used to produce the resistant cell line may be any cancer cell line derived from any cancer.

26. Herein, “stable” has particular meaning in the art of tissue culture and refers to a steady state condition. It is understood that a stable cell line, as used herein, refers to one that maintains its resistance to chemotherapeutics over time. Conversely, an unstable cell or cell line is one in which the cells being used do not maintain resistance over time. Thus, a stable cell line is one that can give rise to multiple generations without loss of resistance of the cell.

27. Thus, an example of a disclosed cell line is a stable CPT-11 resistant cell line, wherein the CPT-11 resistant cell line is a cancer cell line derived from a cancer selected from the group of cancers consisting of lymphomas (Hodgkin's and non-Hodgkin's), B-cell lymphoma, T-cell lymphoma, leukemias, carcinomas, carcinomas of solid tissues, squamous cell carcinomas, adenocarcinomas, sarcomas, gliomas, high grade gliomas, blastomas, plasmacytomas, melanomas, myelomas, AIDS-related lymphomas or sarcomas, metastatic cancers, mycosis fungoides, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, skin cancer, liver cancer, squamous cell carcinomas of the mouth, throat, larynx, and lung, colon cancer, cervical cancer, cervical carcinoma, breast cancer, epithelial cancer, genitourinary cancer, pulmonary cancer, esophageal carcinoma, gall bladder cancer, hematopoietic cancers, testicular cancer, rectal cancers, prostatic cancer, and pancreatic cancer.

28. Optionally, the cancer cell line can be a colon cancer cell line and that cell line can be SW948. More specifically, the cell or cell line of the invention is represented by ATCC Catalog No. CCL-237.

2. Methods of Making the Compositions

29. The compositions disclosed herein and the compositions necessary to perform the disclosed methods can be made using any method known to those of skill in the art for that particular reagent or compound unless otherwise specifically noted.

30. Described herein are methods of making or deriving a cell line that is resistant to a chemotherapeutic agent, for example, CPT or a derivative or a metabolite thereof. Within the scope of this invention is a cell line that is resistant to CPT or a derivative or a metabolite thereof and at least one additional chemotherapeutic agent. Thus, specifically disclosed is a method of deriving a stable cell line resistant to CPT or a derivative or a metabolite thereof, comprising (a) contacting repeatedly a population of cells of a cancer cell line with CPT or the derivative or the metabolite thereof during at least a three month period of time, wherein the cells are contacted with CPT or the derivative or the metabolite thereof in higher concentrations over the period of time and wherein the CPT or the derivative or the metabolite thereof is removed between contacting steps, and (b) selecting cells with resistance, wherein the resistance persists after the contacts with CPT or the derivative or the metabolite thereof are discontinued, cells with persistent resistance being a stable cell line. As used herein, “higher concentrations” means that the concentration of a drug used in the contacting step is greater than the concentration of the drug used in the previous contacting step. It is understood and herein contemplated that the cells can be contacted with CPT or a derivative or a metabolite thereof, for example, CPT-11, 10-OH-CPT or SN38, for time periods other than about three months. Therefore, provided herein are methods comprising contacting repeatedly a population of cells of a cancer cell line with CPT or a derivative or a metabolite thereof during a one, two, three, four, five, six, seven, eight, nine, ten, eleven, or twelve month period of time. For example, specifically disclosed are methods comprising contacting repeatedly a population of cells of a cancer cell line with CPT or a derivative or a metabolite thereof during a three month period of time. Also disclosed are methods comprising contacting repeatedly the cells of a cancer cell line with CPT or a derivative or a metabolite thereof for two, three, four, or five years. It is also understood that the cells can be further resistant to at least one additional chemotherapeutic agent, wherein the additional chemotherapeutic agent is selected from the group consisting of actinomycin D, camptothecin, capecitabine, carboplatin, cisplatin, colchicine, daunorubicin, docetaxel, doxorubicin, epirubicin, etoposide, 5-fluorouracil, gemcitabine, melphalan, methotrexate, mitomycin C, mitoxantrone, paclitaxel, topotecan, vinblastine, and vincristine.

31. The contacting step of the method exposes the cell to the chemotherapeutic (e.g., CPT-11) and involves the periodic contacting of the cells with the chemotherapeutic in fresh culture medium containing fresh chemotherapeutic. Thus, herein disclosed are methods, wherein the contacting step is repeated every one day, every two to three days or less frequently. The contacting step can also be repeated after longer periods of contact; therefore, also disclosed are methods, wherein the contacting step is repeated every 1 week, 2 weeks, 3 weeks or 1 month.

32. To insure the survival of cells exposed to the chemotherapeutic, it may be necessary to remove the contact of the chemotherapeutic with the cells. Thus, following a period of contact with a chemotherapeutic, there is a period of recovery in which no contact between the chemotherapeutic and the cell line occurs. Optionally, the recovery period is approximately equal to the period of contact. It is disclosed and herein contemplated that periods of contact can occur every three to four days, but also can occur for longer periods of 1 week, 2 weeks, 3 weeks or 1 month, or any period in between, or occur for periods as short as everyday. Similarly, it is disclosed and herein contemplated that periods of recovery can occur every three to four days, but also can occur for longer periods of 1 week, 2 weeks, 3 weeks or 1 month, or any period in between, or occur for periods as short as everyday.

33. It is understood that one of ordinary skill in the art will be able to determine through routine techniques whether the period of recoveries can be discontinued or should be maintained throughout for the development of the resistant cell line. As such, disclosed are methods in which the alternating periods of contact and recovery are discontinued after 1 week, 2 weeks, 3 weeks, 1 month, 2 months, or 3 months or longer. Also contemplated are methods wherein the alternating periods of contact and recovery are not discontinued, as well as methods that do not contain any period of recovery. For example, also contemplated are methods wherein the alternating periods of contact and recovery are not discontinued in the first month, as well as methods that do not contain any period of recovery after the first month.

34. In order to increase the resistance to a chemotherapeutic, it may be necessary or desired to increase the concentration used to contact the cells. This determination is well within the abilities and knowledge of one of ordinary skill in the art. An example of one method to make such a determination is to check the inhibitory concentration for 50% colony formation (IC₅₀) of a cell line at an exposure level at various time periods following contact with the chemotherapeutic at a given concentration. If the resistance has stopped increasing, then one of ordinary skill in the art can increase the concentration of the chemotherapeutic during periods of contact. It is understood that a test to determine if the resistance of the cell line has stopped increasing is not required to increase the concentration used to contact the cells. One of ordinary skill in the art may simply decide to increase the concentration after a period of time having conducted no test on the resistance of the cell line. Thus, disclosed and herein contemplated are methods, wherein the concentration of the chemotherapeutic is increased every one week, two weeks, three weeks, one month, two months, three months, four months, five months, or six months or any period in between. Also disclosed are methods in which the concentration is changed after varying periods of contact time or following a determination of the level of resistance. For example, the concentration of CPT-11 can be changed every 1 week, every 2 weeks or every 3 weeks. One can also initially contact a cell line with CPT-11 for two months before increasing the concentration of CPT-11 and then, following the increase, wait only two weeks before increasing the concentration again. As used throughout, selecting cells with resistance to the chemotherapeutic will be understood by one of skill in the art to include any variety of methods. For example, resistance to cells can be selected as those cells that survive contact with the chemotherapeutic at a selected dose.

35. An aspect of the resistant cell lines described herein and the methods of making those cell lines is that the resistance will persist over time. Thus, provided herein are resistant cell lines wherein resistance persists in the cell line for at least about three weeks, one, two, three, four, five, six months or longer or any period in between after contacts with, for example, CPT-11 are discontinued. Also disclosed are methods of making the cell lines, wherein resistance persists in the cell line for at least about three weeks, one, two, three, four, five, six months or longer or any period in between after contacts with, for example, CPT-11 are discontinued.

36. The treatment of SW948CPTH cells with increasing doses of, for example, CPT-11 was continued in order to develop a more resistant cell line useful in an in vivo tumor model to test agents that reverse chemoresistance. For example, the cells are useful when the chemotherapeutic combines with antibodies to cell surface receptors associated with cancers (e.g., EGFR) to demonstrate whether the antibody treatment results in reversal of CPT-11 resistance. For example, the cell line is useful in combination with Erbitux® or other antibodies to demonstrate whether Erbitux® or other antibodies can reverse CPT-11 resistance. Therefore, the cell lines described herein can be used to determine the ability of an antibody or other agent to reverse, for example, CPT-11 resistance. The cell lines resistant to CPT or a derivative or a metabolite thereof and at least one additional chemotherapeutic agent can be used to determine the ability of an antibody or other agent to reverse resistance to the chemotherapeutic agents. It is understood that the cell lines described herein could be used to demonstrate the effectiveness of an antibody to reverse resistance to a chemotherapeutic is not limited to Erbitux®. Other antibodies that can be tested include but are not limited to Herceptin®, Mylotarg®, Orthoclone OKT3®, ReoPro®, Simulect®, Synagis®, Zenapax®, Zevalin™, Abx-CBL, Antegren®, Avastin™, BEC2, Bexxar®, CAT-152, CDp 870, D2E7, Felvizumab, HNK20, HuMax-CD4, Humicade™, ING-1, Atgam®, MabThera®, MDX-210, Oncolym®, OvaRex®, Pemtumomab, Protovir™, Ragavirumab, Xolair, Zamyl®, Xanelim, Segard, Thymoglobulin®, CroFab®, CytoGam®, DigiTab®, Diphtheria Antitoxin, Hepatitis B Immune Globulin, Respigram®, Rho(D) Immuno Globulin, Tetanus Immuno Globulin, Viper-Tab®, CytoTAb®, CEA-Scan®, LeukoScan®, OncoScint®, ProstaScint®, AFP-Scan, Tru-Scint AG, Tru-Scint AD, anti-DR4, anti-DR5 (including TRA-8), ABX-EGF, Anti-VEGF, Campath1H, LymphoCide™, Lym-1, Panorex 17-1A®, Rituxan®, Vitaxin®, Remicade®, and Infliximab. Additionally, the disclosed methods are not limited to antibodies; small molecule tyrosine kinase inhibitors can also be used. Thus, another aspect specifically disclosed and herein contemplated is using the cell lines described herein to determine the ability of a small molecule tyrosine kinase inhibitor to reverse, for example, CPT-11 resistance. Tyrosine kinase inhibitors can include, but are not limited to IRESSA and TARCEVA.

37. Furthermore, resistance to a chemotherapeutic agent is not limited to treatments with CPT, a derivative or a metabolite thereof. Thus, specifically contemplated are methods of using the disclosed cell lines to test the ability of an antibody to reverse resistance to chemotherapeutics including but not limited to actinomycin D, camptothecin, capecitabine, carboplatin, cisplatin, colchicine, daunorubicin, docetaxel, doxorubicin, epirubicin, etoposide, 5-fluorouracil, gemcitabine, melphalan methotrexate, mitomycin C, mitoxantrone, paclitaxel, topotecan, vinblastine, and vincristine. As is demonstrated in Table 1, specifically disclosed are CPT-11 resistant cell lines that have been exposed to increasing concentrations of CPT-11. Thus specifically disclosed are cell lines that have been exposed to 4, 6, 8, 10, 20, 30, 40, and 80 μg/ml of CPT-11. It is understood that the art of varying the concentration of CPT-11 or any other chemotherapeutic is well within the knowledge of the art, and, therefore, herein contemplated and specifically disclosed are cell lines exposed to concentrations of CPT-11 or other chemotherapeutic less than 80 μg/ml or progressing to 80 μg/ml in different steps than 4, 6, 8, 10, 20, 30, 40, and 80 μg/ml. It is also comtemplated that higher concentrations may be used than used in the examples described herein. Thus, provided are cell lines exposed to chemotherapeutics at 100, 110, 140, 150, 160, 200, 320, 500, 1000, and 1280 μg/ml.

TABLE 1
Summary of results of cologenic assay to determine
CPT-11 IC₅₀ dose: SW948CPTH (CPT-11 high) cells were
exposed to different concentrations of CPT-11 for
various periods of time before determination of IC₅₀.

| | Days exposed | | Cells stored after |
|---|---|---|---|
| CPT-11 | to CPT-11 | IC₅₀ | exposure to CPT-11 |
| μg/ml | when assayed | (μg/ml) | (days) |
| 4 | 105 | 12.5 | 105 |
| 6 | 20 | 17.7 | 20 |
| 8 | 14 | 18.2 | 26 |
| 8 | 20 | 15.6 | |
| 10 | 23 | 21.3 | 34 |
| 20 | 25 | 23.6 | |
| 20 | 30 | 18.2 | |
| 20 | 51 | 24 | |
| 20 | 65 | 34 | 65 |
| 20 | 60 | 19* | |
| 20 (samples | 77 | 28.6 | |
| for | | | |
| inoculation) | | | |
| 30 | 20 | 18 | |
| 30 | 19 | 21.3 | |
| 30 | 47 | 19 | 53 |
| 40 | 30 | 55 | 54 |
| 80 | 8 | 79 | 8 |

*CPT-11 was withdrawn for 33 days prior to IC₅₀ determination. All other samples were maintained and tested in the presence of CPT-11 in the culture medium.

38. Throughout this document reference will be made to “Erbitux®,” “IMC-C225,” and “C225.” It is fully intended that these terms refer to the same agent and will be used interchangeably throughout the application.

39. The use of mitoxantrone to produce resistance in a different cancer cell line is known in the art (Brangi et al., Cancer Res 59:5938-5946, 1999). Specifically contemplated is the use of mitoxantrone to establish a CPT-11 resistant SW948 cell line.

40. Also disclosed are animals produced by the process of adding to the animal any of the cells disclosed herein. Thus, also disclosed are in vivo animal models that may be used to screen agents to assess the ability of the agent to reduce or reverse resistance to a chemotherapeutic. For example, specifically contemplated are animals injected with the CPT-11 resistant SW948 cells disclosed herein. Thus, provided herein is an animal comprising SW948CPTH cells. Also disclosed are animals comprising SW948CPTL cells.

3. Methods of Using the Compositions

a) Methods of using the compositions as research tools

41. The disclosed compositions can be used in a variety of ways as research tools. One aspect of the disclosed methods of using the cells and cell lines disclosed herein is a method of screening for an agent that reduces resistance to a chemotherapeutic, thus making a cancer responsive to the chemotherapeutic. The method comprises contacting the resistant cell disclosed herein or a plurality thereof with an agent to be screened and with the chemotherapeutic and detecting reduced cell division in the cell or plurality of cells as compared to a control cell or plurality of cells, or increased cell death indicating an agent that reduces chemotherapeutic resistance. It is contemplated that the contacting step can be in vitro or in vivo. As used herein, a “control cell” can be a cell which is not contacted by the agent to be screened. Thus, provided is a method of screening for an agent that reduces resistance to a selected chemotherapeutic agent, comprising (a) contacting a cell, or a plurality thereof, resistant to CPT or a derivative or a metabolite thereof, wherein the cell is from a stable resistant cell line, with an agent to be screened and with CPT or the derivative or the metabolite thereof to which the cell is resistant, and (b) detecting reduced cell division in the cell or cells as compared to a control cell or cells, reduced cell division indicating an agent that reduces resistance to the chemotherapeutic. The selected chemotherapeutic can be one or more of CPT, CPT-11, 10-OH-CPT, SN38. Also provided is a method of screening for an agent that reduces resistance in a cell or cell line that is resistant to CPT or a derivative or a metabolite thereof and at least one additional chemotherapeutic agent, comprising contacting the resistant cell line disclosed herein with an agent to be screened and with either CPT or a derivative or a metabolite thereof and the additional chemotherapeutic agent and detecting reduced cell division in the cell as compared to a control cell, reduced cell division indicating an agent that reduces resistance of the cell to CPT or a derivative or a metabolite thereof and the additional chemotherapeutic agent(s). The additional chemotherapeutic can be selected from the group consisting of actinomycin D, camptothecin, capecitabine, carboplatin, cisplatin, colchicine, daunorubicin, docetaxel, doxorubicin, epirubicin, etoposide, 5-fluorouracil, gemcitabine, melphalan, methotrexate, mitomycin C, mitoxantrone, paclitaxel, topotecan, vinblastine, and vincristine. Optionally, the cell or cell line is contacted with the agent to be screened and more than one chemotherapeutic to which the cell or cell line is resistant. It is understood and herein contemplated that there are many methods that may be employed to measure reduction of chemoresistance including, but not limited to, counting cells directly, proliferation assays, apoptosis assays, and clonogenic assays.

42. Herein, “reduction” or “reduced” refers to change that occurs compared to a control. The reduction can include, for example, 10%, 20%, 30%, 40%, 50%, 60%, 70%, 80%, 90%, or 100%, or any intermediate percent decrease in the rate of proliferation or in the number of cells relative to a control population and can include, but is not limited to, the ablation of a cell or plurality of cells as well as an interruption in the cell cycle or proliferative arrest of the same. By “reduction in chemoresistance” is meant a 10%, 20%, 30%, 40%, 50%, 60%, 70%, 80%, 90%, or 100% or any intermediate percent decrease in the number of cells in treated cells as compared to a control cell or population thereof. Control cells can include the same cells before or after treatment (e.g., treatment with an agent to be screened) or an untreated population of cells (e.g., a non-chemoresistant parent cell or cell line).

43. Herein, “untreated” refers to a cell, plurality of cells, or cell line that has not been treated with a chemotherapeutic or agent being screened. Untreated can also refer to a cell, plurality of cells, or cell line prior to treatment.

44. A reduction in cell division is not the only manner that can be used to measure the effectiveness of an agent at reducing the resistance of a chemotherapeutic. Increased cell death can be used with equivalent results. Thus, disclosed are methods of screening for an agent that reduces resistance to a chemotherapeutic, comprising contacting the resistant cell disclosed herein or a plurality of cells thereof with an agent to be screened and with a chemotherapeutic and detecting an increased cell death as compared to a control population of cells, increased cell death indicating an agent that reduces chemotherapeutic resistance. For example, the invention provides a method of screening for an agent that reduces CPT-11 resistance by contacting the resistant cell or plurality of cells with an agent to be screened and with CPT-11 and detecting an increased cell death as compared to a control population of cells, increased cell death indicating an agent that reduces CPT-11 resistance. Further provided is a method of screening for an agent that reduces resistance in a cell or cell line that is resistant to CPT or a derivative or a metabolite thereof and at least one additional chemotherapeutic agent, comprising contacting the resistant cell line disclosed herein with an agent to be screened and with either CPT or a derivative or a metabolite thereof and the additional chemotherapeutic agent and detecting increased cell death as compared to a control cell or cells, increased cell death indicating an agent that reduces resistance of the cell or cells to CPT or a derivative or a metabolite thereof and the additional chemotherapeutic agent(s).

45. The art of measuring cell death is well known and any technique used to do so can be used to establish the desired effect. Such methods can include but are not limited to Annexin V, propidium iodide staining, terminal deoxynucleotidyl transferase-mediated dUTp nick-end labeling (TUNEL), caspase assays, DNA laddering, incorporation of tritiated thymidine, and vital dye staining.

46. It is understood that the disclosed methods can be modified to test more complex cancer therapies such as combination therapies involving multiple chemotherapeutics, a chemotherapeutic plus radiation, antibodies plus a chemotherapeutic and/or radiation. Thus, also disclosed are methods of screening for an agent that reduces resistance to a chemotherapeutic, further comprising contacting the cell with one or more chemotherapeutics and/or radiation and/or antibodies in addition to the one to which the cells are resistant. For example, the invention includes contacting CPT-11 resistant cells with the agent being screened, CPT-11 and one or more non-CPT-11 chemotherapeutics, or contacting CPT-11 resistant cells with the agent to be screened, CPT-11, and any combination of other chemotherapeutics, radiation therapy, and/or antibody therapy.

47. Radiation is well known in the treatment of cancers and can have a profound effect on combination therapies. As such, it is understood that to properly assess an agent being screened using the methods described herein, it may be necessary to expose the cells to radiation. Thus, one embodiment of the disclosed methods of screening are methods further comprising treating the cell with a therapeutic amount of radiation.

b) Methods of Treatment

48. Agents identified via the screening methods disclosed herein can be used for the treatment of cancer specifically enhancing the effects of chemotherapeutics. Thus, one embodiment of the disclosed invention is a method of treating a subject with cancer, comprising administering to the subject a therapeutic amount of the agent identified by the disclosed screening methods. Thus, provided herein is a method of treating a subject with cancer, wherein the subject is resistant to CPT or a derivative or a metabolite thereof, comprising administering to the subject a therapeutic amount of the agent identified by the disclosed screening methods. Further, a therapeutic amount of an agent that lowers intracellular pH can be administered to the subject. Examples of agents that lower intracellular pH include, but are not limited to, amiloride and its derivative, 5-(N-ethyl-N-isopropyl)amiloride (EIPA). For example, as taught below in Example 7, amiloride lowers the intracellular pH of CPT-11 resistant SW948CPTH cancer cells, thereby increasing the intracellular amount of the active form of CPT-11 and of a metabolite, SN38, thereby decreasing resistance of the cells to CPT-11. Thus, disclosed are methods of treating a subject with cancer, wherein the subject is resistant to, for example, CPT-11, comprising administering to the subject a therapeutic amount of the agent identified by the disclosed screening methods and further comprising administering to the subject a therapeutic amount of an agent that lowers intracellular pH, for example, amiloride or its derivative 5-(N-ethyl-N-isopropyl)amiloride (EIPA.

49. Also provided herein is a method of reducing resistance in a target cell to a chemotherapeutic, comprising contacting the target cell with an agent that lowers intracellular pH as compared to a control, the lowering in pH reducing resistance in the target cell. As used herein, a “target cell” is a cell from a stable cell line that is resistant to a chemotherapeutic that is further contacted by one or more of CPT or a derivative or a metabolite thereof, an additional chemotherapeutic agent, or an agent that lowers intracellular pH. As used herein, a chemotherapeutic agent that contacts a cell from a stable cell line that is resistant to the chemotherapeutic is a “selected chemotherapeutic.” It is contemplated that the contacting step can be in vitro or in vivo. The chemotherapeutic can be CPT or a derivative or a metabolite thereof or one or more of actinomycin D, camptothecin, capecitabine, carboplatin, cisplatin, colchicine, daunorubicin, docetaxel, doxorubicin, epirubicin, etoposide, 5-fluorouracil, gemcitabine, melphalan, methotrexate, mitomycin C, mitoxantrone, paclitaxel, topotecan, vinblastine, and vincristine.

50. The disclosed compositions can be used to treat any disease where uncontrolled cellular proliferation occurs such as cancers and proliferative diseases such as arthritis or other autoimmune diseases. A non-limiting list of different types of cancers is as follows: lymphomas (Hodgkin's and non-Hodgkin's), B-cell lymphoma, T-cell lymphoma, leukemias, carcinomas, carcinomas of solid tissues, squamous cell carcinomas, adenocarcinomas, sarcomas, gliomas, high grade gliomas, blastomas, neuroblastomas, plasmacytomas, melanomas, myelomas, AIDS-related lymphomas or sarcomas, metastatic cancers, mycosis fungoides, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, prostate cancer, skin cancer, liver cancer, squamous cell carcinomas of the mouth, throat, larynx, and lung, colon cancer, cervical cancer, cervical carcinoma, breast cancer, epithelial cancer, genitourinary cancer, pulmonary cancer, esophageal carcinoma, hematopoietic cancers, testicular cancer, rectal cancers, sarcomas, prostatic cancer, gall bladder cancer, or pancreatic cancer.

4. Antibodies

a) Antibodies Generally

51. The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as described herein. The antibodies are tested for their desired activity using the in vitro assays described herein, or by analogous methods, after which their in vivo therapeutic and/or prophylactic activities are tested according to known clinical testing methods.

52. The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567 and Morrison et al., Proc. Natl. Acad. Sci. USA, 81:6851-6855, 1984).

53. Monoclonal antibodies of the invention can be prepared using hybridoma methods, such as those described by Kohler and Milstein, Nature, 256:495, 1975. In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro, e.g., using the HIV Env-CD4-co-receptor complexes described herein.

54. The monoclonal antibodies may also be made by recombinant DNA methods, such as those described in U.S. Pat. No. 4,816,567 (Cabilly et al.). DNA encoding the monoclonal antibodies of the invention can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

55. In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

56. The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr Opin Biotechnol 3:348-354, 1992).

57. As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods of the invention serves to lessen the chance that an antibody administered to a human will evoke an undesirable immune response.

b) Human Antibodies

58. The human antibodies of the invention can be prepared using any technique. Examples of techniques for human monoclonal antibody production include those described by Cole et al. (Monoclonal Antibodies and Cancer Therapy, Alan R., Ed. Liss, p. 77, 1985) and by Boerner et al. (J Immunol, 147(1):86-95, 1991). Human antibodies of the invention (and fragments thereof) can also be produced using phage display libraries (Hoogenboom et al., J Mol Biol, 227:381, 1991; Marks et al., J Mol Biol, 222:581, 1991).

59. The human antibodies of the invention can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255, 1993; Jakobovits et al., Nature, 362:255-258, 1993; Bruggermann et al., Year in Immunol. 7:33, 1993). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

c) Humanized Antibodies

60. Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an Fc, Fv, Fab, Fab′, or other antigen-binding portion of an antibody) which contains a portion of an antigen binding site from a non-human (donor) antibody integrated into the framework of a human (recipient) antibody.

61. To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525, 1986, Reichmann et al., Nature, 332:323-327, 1988, and Presta, Curr Opin Struct Biol, 2:593-596, 1992).

62. Methods for humanizing non-human antibodies are well known in the art. For example, humanized antibodies can be generated according to the methods of Winter and co-workers (Jones et al., Nature, 321:522-525, 1986, Riechmann et al., Nature, 332:323-327, 1988, Verhoeyen et al., Science, 239:1534-1536, 1988), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5, 939,598 (Kucherlapati et al.), U.S. Pat. No. 6,130,364 (Jakobovits et al.), and U.S. Pat. No. 6,180,377 (Morgan et al.).

d) Administration of Antibodies

63. Antibodies of the invention are preferably administered to a subject in a pharmaceutically acceptable carrier. Suitable carriers and their formulations are described in Remington: The Science and Practice of Pharmacy (19th ed.) A. R. Gennaro, Ed., Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped particles, e.g., films, liposomes or microparticles. It will be apparent to those persons skilled in the art that certain carriers may be more preferable depending upon, for instance, the route of administration and concentration of antibody being administered.

64. The antibodies can be administered to the subject, organ, tissue, or cell by a variety of methods. For example, the antibody can be added to in vitro culture. The antibody can also be administered to a subject, organ, tissue, or cell in situ by injection (e.g., intravenous, intraperitoneal, subcutaneous, intramuscular), or by other methods such as infusion that ensure its delivery to the target in an effective form. Local or intravenous injection is preferred.

65. Effective dosages and schedules for administering the antibodies may be determined empirically, and making such determinations is within the skill in the art. Those skilled in the art will understand that the dosage of antibodies that must be administered will vary depending on, for example, the subject that will receive the antibody, the route of administration, the particular type of antibody used and other drugs being administered. Guidance in selecting appropriate doses for antibodies is found in the literature on therapeutic uses of antibodies, e.g., Handbook of Monoclonal Antibodies, Ferrone et al., eds., Noges Publications, Park Ridge, N.J., 1985 ch. 22 and pp. 303-357; Smith et al., Antibodies in Human Diagnosis and Therapy, Haber et al., eds., Raven Press, New York, 1977 pp. 365-389. A typical daily dosage of the antibody used alone might range from about 1 μg/kg to up to 100 mg/kg of body weight or more per day, depending on the factors mentioned above.

5. Pharmaceutical Carriers/Delivery of Pharmaceutical Products

66. As described above, the compositions can also be administered in vivo in a pharmaceutically acceptable carrier. By “pharmaceutically acceptable” is meant a material that is not biologically or otherwise undesirable, i.e., the material may be administered to a subject, along with the cell, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

67. The compositions may be administered orally, parenterally (e.g., intravenously), by intramuscular injection, by intraperitoneal injection, transdermally, extracorporeally, topically or the like. As used herein, “topical intranasal administration” means delivery of the compositions into the nose and nasal passages through one or both of the nares and can comprise delivery by a spraying mechanism or droplet mechanism, or through aerosolization of the cell. The latter may be effective when a large number of animals is to be treated simultaneously. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the disorder being treated, the particular cell used, its mode of administration and the like. Thus, it is not possible to specify an exact amount for every composition. However, an appropriate amount can be determined by one of ordinary skill in the art using only routine experimentation given the teachings herein.

68. Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

69. The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem, 2:447-451, 1991; Bagshawe, K. D., Br J Cancer, 60:275-281, 1989; Bagshawe, et al., Br J Cancer, 58:700-703, 1988) Senter, et al., Bioconjugate Chem, 4:3-9, 1993; Battelli, et al., Cancer Immunol lmmunother, 35:421-425, 1992; Pietersz and McKenzie, Immunolog. Reviews, 129:57-80,1992; and Roffier, et al., Biochem Pharmacol, 42:2062-2065, 1991). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Res., 49:6214-6220, 1989; and Litzinger and Huang, Biochimica et Biophysica Acta, 1104: 179-187, 1992). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA Cell Biol 10:6, 399-409, 1991).

a) Pharmaceutically Acceptable Carriers

70. The compositions, including antibodies, can be used therapeutically in combination with a pharmaceutically acceptable carrier.

71. Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

72. Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

73. The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies or agents can be administered intravenously, intraperitoneally, intramuscularly, subcutaneously, intracavity, or transdermally.

74. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

75. Formulations for topical administration may include ointments, lotions, creams, gels, drops, suppositories, sprays, liquids, and powders. Conventional pharmaceutical carriers, aqueous, powder or oily bases, thickeners, and the like may be necessary or desirable.

76. Compositions for oral administration include powders or granules, suspensions or solutions in water or non-aqueous media, capsules, sachets, or tablets. Thickeners, flavorings, diluents, emulsifiers, dispersing aids or binders may be desirable.

77. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, tri-alkyl and aryl amines and substituted ethanolamines.

b) Therapeutic Uses

78. The dosage ranges for the administration of the compositions are those large enough to produce the desired effect in which the symptoms of the disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any contraindications. Dosage can vary, and can be administered in one or more dose administrations daily, for one or several days.

79. The cell lines disclosed herein can also be used, for example, as tools to isolate and test new drug candidates to be used in combination therapies for cancer and to overcome resistance to a given therapeutic. For example, the disclosed cell lines can be used as a CPT-11 resistant cell line in which an agent is used in combination with CPT-11 to determine whether the agent will reverse CPT-11 resistance. In another aspect, a disclosed cell line can be used as a cell line resistant to CPT-11 and at least one additional chemotherapeutic agent in which an agent is used in combination with CPT-11 and at least one additional chemotherapeutic agent to determine whether the agent will reverse resistance to CPT-11 and at least one additional chemotherapeutic agent.

### C. EXAMPLES

80. The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

81. SW948 human colon cancer cells were treated with escalating concentrations of CPT-11. FIG. 1 shows the schema for dose escalation of CPT-11. Ninety days following the initiation of treatment, untreated parental cells (SW948) and CPT-11 treated cells (SW948CPTH) were plated. Five hours after plating, CPT-11 was added to each set of cells at 0, 0.5, 1, 2, 4, 8, 16, and 32 μg/ml and incubated for 24 hours. Cells were then washed with PBS and cultured in fresh medium without CPT-11 and a colony formation assay initiated. Ten days later, the surviving fraction of the SW948 and SW948CPTH cells was determined. The results are shown in FIG. 2. The respective CPT-11 IC₅₀ concentrations were 2.8 and 12.5 μg/ml. These results illustrate the development of a CPT-11 resistant colon cancer cell line. The treatment of SW948CPTH cells to increasing doses of CPT-11 was continued in order to develop more resistant cell lines. The results are shown in FIG. 3 (SW948CPTH) and FIG. 4 (SW948CPTL) which illustrate that continued exposure to CPT-11 resulted in cell lines with increased CPT-11 resistance (higher IC₅₀ values). One of these cell lines was used in an in vivo tumor model in combination with antibodies to cell surface receptors associated with cancers (e.g., EGFR) to demonstrate whether or not the antibody treatment resulted in reversal of CPT-11 resistance.

82. The IC₅₀ of untreated SW948 cells was 2.1±0.3 μg/ml. This level of sensitivity was similar to that reported for other cell lines (Kanzawa et al. Cancer Res 50:5919-5924, 1990; Kojima et al. Cancer Res 58:4368-4374, 1998; and Kojima et al. J Clin Invest 101:1789-1796, 1998).

### 2. Example 2

83. The intracellular accumulation and conversion of the prodrug CPT-11 to the active metabolite SN38 by the enzyme carboxylesterase in SW948 cells as well as SW948CPTL or SW948CPTH cells can be evaluated by HPLC analysis using the method described by Danks et al. Clin Cancer Res 5:917-924, 1999. The carboxylesterase activity measurements in SW948, SW48CPTH, and SW948CPTL cell lines are shown in FIG. 5. The SN38 IC₅₀ values of the SW948, SW948CPTH, and SW948CPTL cell lines were 0.2±0.01, 1.5±0.1, and 1.8±0.2 μg/ml, respectively. Additionally, multidrug resistance can be evaluated by measuring the expression level of p-glycoprotein (p-gp) (Juliano et al Biochim Biophys Acta 11:152-162, 1976) and breast cancer resistant protein (BCRP) (Doyle et al Proc Natl Acad Sci U S A 95:15665-15670, 1998) in SW948 and the resistant SW948 cell lines (SW948CPTH and SW948CPTL). It is understood that the disclosed methods can also be used with other cell lines including other colon cancer cell lines. For example, the disclosed methods can be used with LS174T, WiDr, HT-29, SW403, DLD-1, SW480, SNU-C1, Caco-2, and COLO 205 cell lines. The results in FIG. 6A show that intracellular accumulation of CPT-11 in both SW948CPTH and SW948CPTL was lower than the parent cell line SW948. Intracellular CPT-11 in SW948CPTH was lower than SW948CPTL after both cell lines were exposed to similar amounts of CPT-11. Verapamil (Tsuruo et al Cancer Res 43:2905-2910, 1983 and Shrivastava et al Cancer Chemother Pharmacol 42:483-490, 1998) increased the intracellular concentration of CPT-11 in both SW948CPTH and SW948CPTL, with a higher increment on SW948CPTL. Verapamil decreased the CPT-11 IC₅₀ value in SW948CPTL cells but not SW948CPTH cells FIG. 6B). The relative expression levels of p-gp and BCRP were determined by flow cytometry and were higher on both SW948CPTH and SW948CPTL than parental SW948 cells. SW948CPTL cells were shown to express higher protein levels of p-gp than SW948 and SW948CPTH cell lines (FIG. 7). In contrast, SW948CPTH cells were shown to express higher protein levels of BCRP than SW948 and SW948CPTL (FIG. 7). SW948CPTL possesses highest level of p-gp, four times higher than SW948 cells and three times higher than SW948CPTH. SW948CPTH expresses highest level of BCRP, 2.7 times higher than SW948 cells and 1.8 times higher than SW948CPTL.

### 3. Example 3

84. A second line of SW948 cells (SW948CPTL) have been treated with a lower level of CPT-11 change in concentration over time. As shown in Table 2, unlike the SW948CPTH cell line, the SW948CPTL cell line was initially exposed to a CPT-11 concentration of 0.5 μg/ml. This cell line was exposed to increasing doses of CPT-11 as shown in Table 2 which differs from the regimen used to produce the SW948CPTH cell line (Table 1). Over time, the SW948CPTL cell line was exposed to 0.5, 1, 2, 2.5, 3, 3.5, 4, 6, 8, 10, 15, 20, and 40 μg/ml of CPT-11.

85. A comparison of Tables 1 and 2 reveals that the two approaches to CPT-11 exposure produces different results even when cells at the same exposure level are tested. As can be seen from the Tables 1 and 2, cells exposed to 8 μg/ml of CPT-11 for 18-20 days had an IC₅₀ of 4.8 μg/ml in the SW948CPTL cells; however, cells receiving the high dose regimen (SW948CPTH) had an IC₅₀ of 15.6 μg/ml. Thus, the high dose regimen results in cells with a higher resistance than cells treated with the lose dose regimen even when maximum exposure levels and length of exposure at that level are the same.

### 4. Example 4

86. Drug resistance to other topoisomerase I and II inhibitors was examined by determining the IC₅₀ dose of each drug using a cell proliferation assay as shown in Table 3. The SW948CPTH cells showed increased resistance to topoisomerase I inhibitors, CPT-11, CPT, and 10-OH-CPT. No change in SW948CPTH resistance was seen using the topoisomerase I inhibitor, lapachone compared to the parental cell line, SW948. The topoisomerase II inhibitor, etoposide, showed a 2-fold increase in resistance by SW948CPTL cells but no change in resistance by SW948CPTH cells when compared to the parental cell line, SW948.

TABLE 2
Total days of exposure of SW948CPTL cells to different doses of CPT-
11: SW948CPTL (CPT-11 low) cells were exposed to different concen-
trations of CPT-11 for various periods of time before determination
of IC₅₀. Several of these cells are stored frozen. Thus, there are
several different SW948CPTL (CPT-11 low) cell lines.

| [CPT-11], μg/ml, | Total days | IC₅₀ | Days at this dose when |
|---|---|---|---|
| before test | at this dose | (μg/ml) | tested |
| 0.5 | 6 | | |
| 1 | 20 | | |
| 2 | 34 | | |
| 2.5 | 12 | | |
| 3 | 14 | | |
| 3.5 | 16 | | |
| 4 | 14 | | |
| 6 | 14 | | |
| 8 | 18 | 4.8 | 7 |
| 10 | 35 | | |
| 15 | 40 | | |
| 20 | 60 | | |
| 40 | 25 | 12.9 | 25 |
| 40 | 55 | 16 | 55 |

TABLE 3
Sensitivity of SW948, SW948CPTH, and SW948CPTL to various drugs
expressed as IC₅₀ in μg/ml. The SW948CPTH cells were treated with
20 μg/ml CPT-11 for 62 days, then the drug was removed and cross-
resistance studies were performed on cells greater than 22 days
without exposure to CPT-11. The SW948CPTL cells were treated with
40 μg/ml CPT-11 for 42 days, then the drug was removed and cross-
resistance studies were performed on cells greater than 28 days
without exposure to CPT-11. Results are expressed as mean ± SD
from 2-4 independent assays done in quadruplicate.

| Drug | SW948 | SW948CPTH | SW948CPTL |
|---|---|---|---|
| CPT-11 | 3.0 ± 0.4 | 43.7 ± 8.6 | ND (not determined) |
| CPT | 0.1 ± 0.1 | 0.6 ± 0.6 | ND |
| 10-OH- | 0.4 ± 0.3 | 2.1 ± 1.0 | ND |
| CPT | | | |
| Lapachone | 1.1 ± 0.2 | 0.8 ± 0.2 | ND |
| Etoposide | 0.9 ± 0.1 | 0.9 ± 0.1 | 1.8 ± 0.3 |

### 5. Example 5

87. The stability of the drug-resistant cancer cell lines was confirmed by removing the selective agent, in this case CPT-11, for an extended period of time and testing for retention of drug resistance. The SW948CPTH cell line was exposed to the drug, CPT-11 (20 μg/ml) for 62 days; then the drug was removed for up to 122 days. Periodically, the IC₅₀ dose for CPT-11 was tested using a standard clonogenic assay. The data shown in Table 4 indicate the stability of CPT-11 drug resistance in the SW948CPTH cell line.

TABLE 4
Testing the stability of CPT-11 drug resistance in SW948CPTH cells
treated with 20 μg/ml CPT-11 for 62 days by using a clonogenic
assay to determine the IC₅₀ dose to CPT-11 at various days after
removal of CPT-11 from the cell culture medium. SW948 cell line has
an IC₅₀ dose for CPT-11 of 2.1 ± 0.3 μg/ml.

| | Days after | IC₅₀ Dose |
|---|---|---|
| Cell Line | Removal of CPT-11 | (μg/ml) |
| SW948CPTH | 5 | 25.0 |
| SW948CPTH | 21 | 18.9 |
| SW948CPTH | 40 | 19.0 |
| SW948CPTH | 45 | 19.2 |
| SW948CPTH | 68 | 11.4 |
| SW948CPTH | 89 | 13.4 |
| SW948CPTH | 122 | 17.8 |

### 6. Example 6

88. The sensitivity of cell lines to CPT-11 which were established from four SW948CPTH tumor xenografts at 134, 121, 124, or 103 days after tumor cell injection is shown in Table 5. Table 5 shows the CPT-11 IC₅₀ values for the SW948CPTH cell lines recovered from xenografts as described in paragraph 16. After recovery of the cells from the xenograft, the clonogenic assay showed the stability of CPT-11 drug resistance in the SW948CPTH cell lines derived from the SW948CPTH xenografts.

TABLE 5
Testing the stability of CPT-11 drug resistance in SW948CPTH cells
recovered from tumor xenografts (FIG. 12). The tumors were removed
103-134 days after tumor cell implant. The tumors were minced in
trypsin-EDTA buffer and the individual cells were cultured for 2-4
weeks in CPT-11-free culture medium. The CPT-11 IC₅₀ dose was
determined using a clonogenic assay.

| | Days after | CPT-11 IC₅₀ Dose |
|---|---|---|
| Cell line from xenograft | implant | (μg/ml) |
| SW948CPTH xenograft #16 | 134 | 24.7 |
| (CPT-11 treated tumor) | | |
| SW948CPTH xenograft #51 | 121 | 18.0 |
| (untreated tumor) | | |
| SW948CPTH xenograft #53 | 124 | 32.2 |
| (untreated tumor) | | |
| SW948CPTH xenograft #54 | 103 | 25.1 |
| (untreated tumor) | | |

### 7. Example 7

89. Characteristics of SW948CPTH and SW948CPTL: Resistant cell lines SW948CPTH and SW948CPTL were obtained at different starting doses of CPT-11. The characteristics of these two cell lines are different based on following tests:
1) Resistance to CPT-11: The resistance to CPT-11 was higher on SW948CPTH than SW948CPTL. The IC₅₀ of SW948CPTH was close to the dose of CPT-11 at which the cells were exposed. However, the IC₅₀ of SW948CPTL was lower than the dose of CPT-11 at which the cells were exposed before the tests.
2) Expression levels of p-gp and BCRP: The expression level of p-gp was highest on SW948CPTL, 4 times higher than SW948 parent cells. However, the expression level of BCRP was highest on SW948CPTH cells. This may indicate a different underlying mechanism of the resistance to CPT-11.
3) Responses to verapamil: Verapamil blocked the efflux of CPT-11 from the SW948CPTH cells and increased the intracellular concentration of CPT-11 to a level similar to the SW948 parent cells. Verapamil increased the intracellular concentration of CPT-11 on SW948CPTL 2.4 times higher than SW948 parent cells. Verapamil reduced the IC₅₀ value of CPT-11 in the SW948CPTL cell line.
4) Amiloride (an inhibitor of the Na⁺/H⁺ antiporter) lowered the intracellular pH of the CPT-11 resistant SW948CPTH cells. Lower intracellular pH increases the intracellular amount of the active form of CPT-11 and of a metabolite, SN38, thereby decreasing resistance of the cells to CPT-11. Thus, modulation of intracellular pH is a possible mechanism for altering resistance of the cells to CPT-11. For example, lowering the intracellular pH of the SW948CPTH cells from about 6.98 to about 6.85 (FIG. 8A) decreased the resistance of the cells to CPT-11 (FIG. 8B). Also, 5-(N-ethyl-N-isopropyl)amiloride (EIPA) decreased the resistance of cells to CPT-11 (FIG. 8B).

5) Cell cycle analysis showed that CPT-11 increased the percentage of cells in the G2-M phase and decreased the percentage in the G0-G1 phase on SW948 parent cell line in a dose dependent manner. No obvious changes were observed on S phase. In contrast, similar amount of CPT-11 did not cause any change of cell cycle on both SW948CPTH and SW948CPTL (Table 6).

TABLE 6
Cell cycle analysis of SW948, SW948CPTH, and SW948CPTL
cells exposed to various concentrations of CPT-11.

| [CPT-11], μg/ml | 0 | 1 | 2 | 5 |
|---|---|---|---|---|
| SW948 | | | | |
| G0-G1(%) | 52.3 ± 2.5 | 36.0 ± 3.2 | 22.1 ± 2.0 | 17.0 ± 1.9 |
| G2-M(%) | 10.3 ± 1.7 | 24.4 ± 1.4 | 35.8 ± 5.1 | 41.0 ± 2.5 |
| S(%) | 37.4 ± 3.6 | 39.6 ± 3.8 | 42.1 ± 6.7 | 42 ± 2.5 |
| SW948CPTH | | | | |
| G0-G1(%) | 45.6 ± 5.8 | 45.7 ± 6.2 | 44.6 ± 3.8 | 45.0 ± 3.0 |
| G2-M(%) | 7.7 ± 4.4 | 8.3 ± 3.1 | 8.9 ± 1.9 | 12.2 ± 2.3 |
| S(%) | 46.7 ± 3.5 | 46 ± 7.5 | 46.5 ± 3.9 | 42.8 ± 4.9 |
| SW948CPTL | | | | |
| G0-G1(%) | 48.9 ± 6.2 | 50.0 ± 1.7 | 45.6 ± 4.0 | 45.3 ± 6.7 |
| G2-M(%) | 8.8 ± 5.0 | 10.0 ± 1.4 | 11.2 ± 2.6 | 13.9 ± 2.2 |
| S(%) | 42.3 ± 10.6 | 40.3 ± 2.4 | 43.1 ± 6.5 | 40.8 ± 6.4 |

### 8. Example 8

90. The SW948CPTH cells that have been treated with CPT-11 were exposed to an increased concentration of CPT-11 equal to 6 μg/ml at 90 days post initiation of treatment. These cells were incubated with CPT-11 at 0, 0.5, 1, 2, 4, 8, 16, and 32 μg/ml for 24 hours. The cells were then washed, fresh medium added, and plated for colony formation. The results are presented in Table 1. They indicate that the IC₅₀ value for the SW948CPTH cells increased from 12.5 μg/ml to 17.7 μg/ml.

91. Additional studies against SW948 colon cancer cells have been carried out in vitro and with SW948 tumor xenografts. The effects of Erbitux®, CPT-11, and radiation on the proliferation of SW948 cells is shown in FIG. 9. The treatments that produced the greatest inhibition of proliferation were Erbitux®+CPT-11 and Erbitux®+CPT-11+radiation. FIG. 10 illustrates the induction of apoptosis in SW948 cells in vitro following treatment with these agents. CPT-11 treatment resulted in a high level of apoptosis which was not increased any further by combination treatment with Erbitux® and radiation. In the initial therapy study against SW948 xenografts, animals received Erbitux® every 3 days for 6 weeks in combination with radiation every 6 days and CPT-11 (40 mg/kg) every 6 days at 1 hour prior to radiation treatment. The tumor growth curves are shown in FIG. 11. The results illustrate a significantly greater reduction in tumor size and time-to-tumor doubling time after treatment with Erbitux®+CPT-11+radiation (3/7 complete regressions with 2 recurrences) as compared to treatment with Erbitux®+radiation (1/7 complete regressions with recurrence) or CPT-11 treatment (0/7 complete regressions). An additional experiment was carried out with SW948 xenografts in which animals received Erbitux®, CPT-11 (25 mg/kg), and 2 Gy radiation every 3-4 days for a total of 6 doses. The results are shown in FIG. 12. The greatest tumor growth inhibition occurred in the groups that received CPT-11+radiation and Erbitux®+CPT-11+radiation. There were 6/7 complete regressions with 3 recurrences in the Erbitux®+CPT-11+radiation group and 5/7 complete regressions with no recurrences in the CPT-11+radiation group. This experiment was run concurrently with a third experiment in which SW948CPTH cells were used in the xenograft (FIG. 13). As in the other two experiments, the greatest growth inhibition occurred in the Erbitux®+CPT-11+radiation group with 4/7 complete regressions with 1 recurrence.

### 9. Example 9

92. Balb/c athymic nude mice were injected subcutaneously with 2×10⁷ SW948 or SW948CPTH cells. In the initial study with SW948 xenografts that were 71.8±39.8 mm² in size, animals received Erbitux® every 3 days for 6 weeks in combination with radiation every 6 days and CPT-11 (40 mg/kg) every 6 days at 1 hour prior to radiation treatment. In the next study, on day 24, when the SW948 tumors were well established (58.0±29.3 mm²), the animals were randomized into different treatment groups and groups of 7 mice received 1 mg C225 intraperitoneally with additional injections on days 27, 31, 34, 38, and 41. Groups of mice received 25 mg CPT-11 intravenously on days 25, 28, 32, 35, 39, and 42. Groups of mice received 2 Gy ⁶⁰Co radiation to the tumor on days 25, 28, 32,35,39, and 42 1 hour after CPT-11 injection. Change in average tumor size measured with calipers (surface area equal to product of two largest diameters) for each group relative to the size on day 24 was determined. Similarly, on day 22, when the SW948CPTH tumors were well established (62.6±26.6 mm²), the animals were randomized into different treatment groups and groups of 7 mice received 1 mg C225 intraperitoneally with additional injections on days 26, 29, 33, 36, and 40. Groups of mice received 25 mg/kg CPT-11 intravenously on days 23, 27, 30, 34, 37, and 41, followed 1 hour later by 2 Gy ⁶⁰Co radiation to the tumor. Change in average tumor size relative to day 22 was monitored as described above for SW948 tumors.

93. Throughout this application, various publications are referenced. The disclosures of these publications in their entireties are hereby incorporated by reference into this application in order to more fully describe the state of the art to which this invention pertains. The references disclosed are also individually and specifically incorporated by reference herein for the material contained in them that is discussed in the sentence in which the reference is relied upon.

94. It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

D. References
Doyle, L A et al. A multidrug resistance transporter from human MCF-7 breast cancer cells. Proc Natl Acad Sci USA 1998; 95:15665-15670.
Grizzle W E et al. Factors Affecting Immunohistochemical Evaluation of Biomarker Expression in Neoplasia John Walker's Methods in Molecular Medicine—Tumor Marker Protocols, (Eds. Margaret Hanausek and Zbig-niew Walaszek), Humana Press, Inc., Totowa, N.J., 1998; 161-179.
Grizzle W E et al. Immunohistochemical Evaluation of Biomarkers in Prostatic and Colorectal Neoplasia. John Walker's Methods in Molecular Medicine—Tumor Marker Protocols, (Eds. Margaret Hanausek and Zbigniew Walaszek), Humana Press, Inc., Totowa, N.J., 1998; 143-160.
Juliano, R et al. A surface glycoprotein modulating drug permeability in Chinese hamster ovary cell mutants. Biochim Biophys Acta 1976; 455:152-162.
Kanazawa, F et al. Establishment of a Camptothecin analogue (CPT-11)-resistant cell Line of human non-small cell lung cancer: Characterization and mechanism of resistance. Cancer Res 1990; 50: 5919-5924.
Kojima, A et al. Reversal of CPT-11 resistance of lung cancer cells by adenovirus-mediated gene transfer of the human carboxylesterase cDNA. Cancer Res 1998; 58: 4368-4374.
Pergo, P et al. Ovarian cancer cisplatin-resistant cell lines: Multiple changes including collateral sensitivity to Taxol. Annals Oncol 1998; 9: 423-430.
Prewett, M C et al. Enhanced antitumor activity of anti-epidermal growth factor receptor monoclonal antibody IMC-C225 in combination with Irinotecan (CPT-11) against human colorectal tumor xenografts. Clin Cancer Res 2002; 8: 994-1003.
Shrivastava P et al. Circumvention of multidrug resistance by a quinoline derivative, MS-209, in multidrug-resistant human small-cell lung cancer cells and its synergistic interaction with cyclosporin A or verapamil. Cancer Chemother Pharmacol 1998; 42: 483-490.
Tsuruo T et al. Circumvention of vincristine and adriamycin resistance in vitro and in vivo by calcium influx blockers. Cancer Res 1983; 43: 2905-2910.

What is claimed is:

1. A cell resistant to CPT or a derivative or a metabolite thereof, wherein the cell is from a stable resistant cell line.

2. The cell of claim 1, wherein the cell is resistant to CPT-11.

3. The cell of claim 1, wherein the cell is resistant to 10-OH-CPT.

4. The cell of claim 1, wherein the cell is resistant to SN38.

5. The cell of claim 1, wherein the cell is resistant to at least one additional chemotherapeutic agent.

6. The cell of claim 5, wherein the additional chemotherapeutic agent is one or more selected from the group consisting of actinomycin D, camptothecin, capecitabine, carboplatin cisplatin, colchicine, daunorubicin, docetaxel, doxorubicin, epirubicin, etoposide, 5-fluorouracil, gemcitabine, melphalan, methotrexate, mitomycin C, mitoxantrone, paclitaxel, topotecan, vinblastine, and vincristine.

7. The cell of claim 1, wherein the stable resistant cell line is derived from a cancer cell line.

8. The cell of claim 7, wherein the stable resistant cell line is a cancer cell line derived from a cancer selected from the group of cancers consisting of lymphomas (Hodgkin's and non-Hodgkin's), B cell lymphoma, T cell lymphoma, leukemias, carcinomas, carcinomas of solid tissues, squamous cell carcinomas, adenocarcinomas, sarcomas, gliomas, high grade gliomas, blastomas, plasmacytomas, melanomas, myelomas, AIDS-related lymphomas or sarcomas, metastatic cancers, mycosis fungoides, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, skin cancer, liver cancer, squamous cell carcinomas of the mouth, throat, larynx, and lung, colon cancer, cervical cancer, cervical carcinoma, breast cancer, epithelial cancer, genitourinary cancer, pulmonary cancer, esophageal carcinoma, hematopoietic cancers, testicular cancer, rectal cancers, prostatic cancer, gall bladder cancer and pancreatic cancer.

9. The cell of claim 8, wherein the cancer cell line is a colon cancer cell line.

10. The cell of claim 9, wherein the colon cancer cell line is SW948.

11. A method of deriving a stable cell line resistant to CPT or a derivative or a metabolite thereof, comprising
(a) contacting repeatedly a population of cells of a cancer cell line with CPT or the derivative or the metabolite thereof during at least a three month period of time, wherein the cells are contacted with CPT or the derivative or the metabolite thereof in higher concentrations over the period of time and wherein the CPT or the derivative or the metabolite thereof is removed between contacting steps; and
(b) selecting cells with resistance, wherein the resistance persists after the contacts with CPT or the derivative or the metabolite thereof are discontinued, cells with persistent resistance being a stable resistant cell line.

12. The method of claim 11, wherein the cells are contacted with CPT-11.

13. The method of claim 11, wherein the cells are contacted with 10-OH-CPT.

14. The method of claim 11, wherein the cells are contacted with SN38.

15. The method of claim 11, wherein the cells are further resistant to at least one additional chemotherapeutic agent.

16. The method of claim 15, wherein the additional chemotherapeutic agent is selected from the group consisting of actinomycin D, camptothecin, capecitabine, carboplatin, cisplatin, colchicine, daunorubicin, docetaxel, doxorubicin, epirubicin, etoposide, 5-fluorouracil, gemcitabine, melphalan, methotrexate, mitomycin C, mitoxantrone, paclitaxel, topotecan, vinblastine, and vincristine.

17. The method of claim 11, wherein the contacting step is repeated every two to four days.

18. The method of claim 11, wherein the concentration is increased at least 20 μg/ml when the cells are contacted with CPT-11 over the period of time.

19. The method of claim 11, wherein the resistance persists in the cell line for at least 21 days after the contacting step is discontinued.

20. The method of claim 11, wherein the resistance persists in the cell line for at least two months after the contacting step is discontinued.

21. The method of claim 11, wherein the resistance persists in the cell line for at least three months after the contacting step is discontinued.

22. A cell line derived by the method of claim 11.

23. A method of screening for an agent that reduces resistance to a selected chemotherapeutic agent, comprising (a) contacting the cell of claim 1 or a plurality thereof with an agent to be screened and with the chemotherapeutic to which the cell is resistant; and (b) detecting reduced cell division in the cell or cells as compared to a control cell or cells, reduced cell division indicating an agent that reduces resistance to the chemotherapeutic.

24. The method of claim 23, wherein the selected chemotherapeutic is CPT or a derivative or a metabolite thereof.

25. The method of claim 24, wherein the selected chemotherapeutic is CPT-11.

26. The method of claim 24, wherein the selected chemotherapeutic is 10-OH-CPT.

27. The method of claim 24, wherein the selected chemotherapeutic is SN38.

28. The method of claim 23, wherein the selected chemotherapeutic is actinomycin D, camptothecin, capecitabine, carboplatin, cisplatin, colchicine, daunorubicin, docetaxel, doxorubicin, epirubicin, etoposide, 5-fluorouracil, gemcitabine, melphalan, methotrexate, mitomycin C, mitoxantrone, paclitaxel, topotecan, vinblastine, or vincristine.

29. The method of claim 23, further comprising treating the cell with a therapeutic amount of radiation.

30. The method of claim 23, wherein the contacting step occurs in vitro.

31. The method of claim 23, wherein the contacting step occurs in vivo.

32. A method of treating a subject with cancer, comprising administering to the subject a therapeutic amount of an agent identified by the method of claim 23.

33. The method of claim 32, further comprising administering to the subject a therapeutic amount of an agent that lowers intracellular pH.

34. The method of claim 33, wherein the agent is amiloride or 5-(N-ethyl-N-isopropyl)amiloride (EIPA).

35. The method of claim 32, wherein the subject is resistant to CPT or a derivative or a metabolite thereof, comprising administering to the subject a therapeutic amount of an agent identified by the method of claim 23.

36. The method of claim 35, further comprising administering to the subject a therapeutic amount of an agent that lowers intracellular pH.

37. The method of claim 36, wherein the agent is amiloride or 5-(N-ethyl-N-isopropyl)amiloride (EIPA).

38. A method of reducing resistance in a target cell to a chemotherapeutic, comprising contacting the target cell with an agent that lowers intracellular pH as compared to a control, the lowering in pH reducing resistance in the target cell.

39. The method of claim 38, wherein the chemotherapeutic is CPT.

40. The method of claim 38, wherein the chemotherapeutic is CPT-11.

41. The method of claim 38, wherein the chemotherapeutic is 10-OH-CPT.

42. The method of claim 38, wherein the chemotherapeutic is SN38.

43. The method of claim 38, wherein the chemotherapeutic is one or more selected from the group consisting of actinomycin D, camptothecin, capecitabine, carboplatin, cisplatin, colchicine, daunorubicin, docetaxel, doxorubicin, epirubicin, etoposide, 5-fluorouracil, gemcitabine, melphalan, methotrexate, mitomycin C, mitoxantrone, paclitaxel, topotecan, vinblastine, and vincristine.

44. The method of claim 38, wherein the contacting step is in vitro.

45. The method of claim 38, wherein the contacting step is in vivo.

46. The method of claim 38, wherein the agent is amiloride or 5-(N-ethyl-N-isopropyl)amiloride (EIPA).

47. A method of screening for an agent that reduces resistance to a selected chemotherapeutic, comprising contacting a plurality of cells of claim 1 with an agent to be screened and with the selected chemotherapeutic and detecting an increased cell death as compared to a control population of cells, increased cell death indicating an agent that reduces resistance to the selected chemotherapeutic.

48. The method of claim 47, wherein the selected chemotherapeutic is CPT.

49. The method of claim 47, wherein the selected chemotherapeutic is CPT-11.

50. The method of claim 47, wherein the selected chemotherapeutic is 10-OH-CPT.

51. The method of claim 47, wherein the selected chemotherapeutic is actinomycin D, camptothecin, capecitabine, carboplatin, cisplatin, colchicine, daunorubicin, docetaxel, doxorubicin, epirubicin, etoposide, 5-fluorouracil, gemcitabine, melphalan, methotrexate, mitomycin C, mitoxantrone, paclitaxel, topotecan, vinblastine, or vincristine.

52. The method of claim 47, further comprising contacting the cell with one or more selected chemotherapeutics.

53. The method of claim 47, further comprising treating the cell with a therapeutic amount of radiation.

54. The method of claim 47, wherein the contacting step occurs in vitro.

55. The method of claim 47, wherein the contacting step occurs in vivo.

56. A method of treating a subject with cancer, comprising administering to the subject a therapeutic amount of an agent identified by the method of claim 47.

57. The method of claim 56, further comprising administering to the subject a therapeutic amount of an agent that lowers intracellular pH.

58. The method of claim 57, wherein the agent is amiloride or 5-(N-ethyl-N-isopropyl)amiloride (EIPA).

59. The method of claim 56, wherein the subject is resistant to CPT or a derivative or a metabolite thereof, comprising administering to the subject a therapeutic amount of an agent identified by the method of claim 47.

60. The method of claim 59, further comprising administering to the subject a therapeutic amount of an agent that lowers intracellular pH.

61. The method of claim 60, wherein the agent is amiloride or 5-ethyl-N-isopropyl)amiloride (EIPA).
